(19) United States
(12) Patent Application Publication
(10) Pub. No.: US 2024/0358388 A1
(43) Pub. Date: Oct. 31, 2024

(54) VASCULAR OBSTRUCTION RETRIEVAL DEVICE HAVING SLIDING CAGES PINCH MECHANISM

(71) Applicant: Neuravi Limited, Galway (IE)

(72) Inventors: Thomas O’MALLEY, Westport (IE); Stephen WHELAN, Galway (IE)

(73) Assignee: Neuravi Limited, Galway (IE)

(21) Appl. No.: 18/768,296

(22) Filed: Jul. 10, 2024

Related U.S. Application Data
(63) Continuation of application No. 17/205,179, filed on Mar. 18, 2021, now Pat. No. 12,064,130.

(51) Int. Cl. A61B 17/221; A61B 17/00

(52) U.S. Cl. CPC A61B 17/221; A61B 2017/00367; A61B 2017/00862; A61B 2017/00867

(57) ABSTRACT

The disclosed technology includes a clot retrieval device being configured to retrieve a clot from a blood vessel and having a constrained delivery configuration and a clot engaging configuration. The device can include a first expandable framework having a first plurality of struts that form a first body and a second expandable framework having a second plurality of struts that form a second body upon the clot retrieval device transitioning from the constrained delivery configuration to the clot engaging configuration. In the clot engaging configuration, the first body can be configured to move from a first position to a second position in relation to the second body. Upon moving from the first position to the second position, the clot retrieval device can pinch the clot between the first body and the second body.

### CROSS REFERENCE TO RELATED APPLICATION

The present application is a continuation application of U.S. patent application Ser. No. 17/205,179 filed Mar. 18, 2021. The entire contents of which are hereby incorporated by reference.

### FIELD OF INVENTION

The present disclosure relates generally to devices and methods for removing blockages from blood vessels during intravascular medical treatments.

### BACKGROUND

Clot retrieval devices are used in mechanical thrombectomy for endovascular intervention, often in cases where patients are suffering from conditions such as acute ischemic stroke (AIS), myocardial infarction (MI), and pulmonary embolism (PE). Acute obstructions can include a clot, misplaced devices, migrated devices, large emboli, and the like. Thromboembolism occurs when part or all of a thrombus breaks away from the blood vessel wall. This clot (now called an embolus) is then carried in the direction of blood flow. An ischemic stroke can result if the clot lodges in the cerebral vasculature. A pulmonary embolism can result if the clot originates in the venous system or in the right side of the heart and lodges in a pulmonary artery or branch thereof. Clots can also develop and block vessels locally without being released in the form of an embolus—this mechanism is common in the formation of coronary blockages.

There are significant challenges associated with designing clot retrieval devices that can deliver high levels of performance. First, there are a number of access challenges that make it difficult to deliver devices. In cases where access involves navigating the aortic arch (such as coronary or cerebral blockages) the configuration of the arch in some patients makes it difficult to position a guide catheter. These difficult arch configurations are classified as either type 2 or type 3 aortic arches with type 3 arches presenting the most difficulty.

The tortuosity challenge is even more severe in the arteries approaching the brain. For example, it is not unusual at the distal end of the internal carotid artery that the device will have to navigate a vessel segment with a 180° bend, a 90° bend, and a 360° bend in quick succession over a few centimeters of vessel. In the case of pulmonary embolisms, access is through the venous system and then through the right atrium and ventricle of the heart. The right ventricular outflow tract and pulmonary arteries are delicate vessels that can easily be damaged by inflexible or high profile devices. For these reasons it is desirable that the clot retrieval device be compatible with as low profile and flexible a guide catheter as possible.

Second, the vasculature in the area in which the clot can be lodged is often fragile and delicate. For example, neurovascular vessels can be more fragile than similarly sized vessels in other parts of the body and can be in a soft tissue bed. Excessive tensile forces applied on these vessels could result in perforations and hemorrhage. Pulmonary vessels can be larger than those of the cerebral vasculature, but are also delicate in nature, particularly more distal vessels.

Additionally, the clot can have any of a range of morphologies and consistencies. For example, the clot can be difficult to grip and improper grip can lead to fragmentation which can cause embolization. Long strands of softer clot material can also tend to lodge at bifurcations or trifurcations, resulting in multiple vessels being simultaneously occluded over significant lengths. More mature and organized clot material can be less compressible than softer fresher clot, and under the action of blood pressure it can distend the compliant vessel in which it is lodged. Furthermore, the properties of the clot can be significantly changed by the action of the devices interacting with it. In particular, compression of a blood clot can cause dehydration of the clot and can result in a dramatic increase in both clot stiffness and coefficient of friction.

Lastly, traditional clot retrieval devices employing a pinch mechanism to capture a clot can require the delivery microcatheter to be forward post deployment of the clot retrieval device in order to effectively pinch a clot using the device. However, this can add an additional step in the procedure, thereby resulting in a potentially cumbersome and non-optimal procedure. Due to the critical nature of such procedures, it can be critical to capture a clot in a timely and effective manner.

The challenges described above need to be overcome for devices to provide a high level of success in removing clot and restoring flow.

### SUMMARY

It is desirable for a clot retrieval device to remove a clot from cerebral arteries in patient suffering from AIS, from coronary native or graft vessels in patients suffering from MI, and from pulmonary arteries in patients from PE and from other peripheral arterial and venous vessels in which a clot is causing at least a partial occlusion. Example devices and methods presented herein may be suitable for at least some of such procedures and/or similar procedures.

An example clot retrieval device can have a constrained delivery configuration and a clot engaging configuration and can be configured to remove a clot from a blood vessel. The device can include a first expandable framework having a first plurality of struts that form a first body and a second expandable framework having a second plurality of struts that form a second body. In the clot engaging configuration, the first body can be configured to move from a first position to a second position in relation to the second body.

The first body can have a first inner diameter and the second body can have a second inner diameter. The first inner diameter and the second inner diameter can be substantially equal.

When the first body is in the first position, the first plurality of struts and the second plurality of struts can be disengaged such that a plurality of clot reception spaces are formed.

When the first body is in the second position, the first plurality of struts and the second plurality of struts can be engaged such that an average cross-sectional area of the plurality of clot reception spaces decreases upon movement of the first body from the first position to the second position.

The first plurality of struts can include a radially extending strut and the second plurality of struts can include an eyelet through which the radially extending strut radially extends. The eyelet and the radially extending strut can be configured such that when the first body moves from the first position to the second position, the radially extending strut engages the eyelet to inhibit the first plurality of struts from moving, in relation to the second plurality of struts, beyond the second position. Each eyelet can be tapered.

The clot retrieval device can include a polymer coating to engage the first plurality of struts and the second plurality of struts. The polymer coating can be configured to fail, thereby allowing the first body to move from the first position to the second position.

At least one polymer membrane can be affixed to the first plurality of struts and the second plurality of struts such that the polymer membrane is disposed between the first body and the second body.

The at least one polymer membrane can be in a folded configuration when the first body is in the first position and the at least one polymer membrane can transition to a stretched configuration when the first body moves proximally to the second position.

The clot retrieval device can include a third expandable framework having a third framework of struts that form a third body. The first body and the second body can at least partially surround the third body in the clot engaging configuration.

A proximal end of the clot retrieval device can include a plurality of expanded struts that form a collar.

The third framework of struts can include at least one disconnected strut.

The third body can include a plurality of clot reception spaces. The plurality of clot reception spaces can be configured to engage the clot.

Another example clot retrieval device can have a constrained delivery configuration and a clot engaging configuration and can be configured to remove a clot from a blood vessel. The device can include an inner expandable framework, an outer expandable framework, and a spring. The inner expandable framework can be affixed to a pull wire and can include a first plurality of struts that form an inner body. The outer expandable framework can be affixed to the pull wire and can include a second plurality of struts that form an outer body at least partially surrounding the inner body. The spring can be affixed to a distal end of the pull wire and can have a compressed configuration and an elongated configuration. In the clot engaging configuration, the inner body can be configured to move from a first position to a second position in relation to the outer body such that the spring transitions from the compressed configuration to the elongated configuration.

The outer expandable framework can include a plurality of clot reception spaces that are configured to pinch the clot between the inner body and the outer body when the inner body moves from the first position to the second position.

An example method to capture a clot can include deploying a clot retrieval device proximate the clot where the clot retrieval device includes a first expandable framework forming a first body and a second expandable framework forming a second body at least partially surrounding the first body. The method can further include moving the first body in relation to the second body to pinch at least a portion of the clot between the first body and the second body and capturing one or more fragments of the clot.

Moving the first body in relation to the second body to pinch at least a portion of the clot between the first body and the second body can include applying tension to a pull wire where the pull wire is in mechanical communication with the first body.

The method can further include retracting the first body and the second body simultaneously.

The clot retrieval device can further include a third expandable framework having a third plurality of struts that form a third body. The first body and the second body can at least partially surround the third body. In such configuration, the method can further include retracting the third body in a proximal direction to engage the first body and the third body.

### BRIEF DESCRIPTION OF THE DRAWINGS

FIG. 1A is a side view of an example clot retrieval device in a clot engaging configuration, in accordance with the present disclosure.

FIG. 1B is an additional side view from a different perspective of the clot retrieval device of FIG. 1A, in accordance with the present disclosure.

FIG. 1C is a cross-section view of a shaft of the clot retrieval device of FIGS. 1A and 1B, in accordance with the present disclosure.

FIG. 1D is an expanded view of the pull wire joined to a first plurality of struts of the clot retrieval device of FIGS. 1A and 1B, in accordance with the present disclosure.

FIG. 1E is an expanded view of a radially extending strut and an eyelet of the clot retrieval device of FIGS. 1A and 1B, in accordance with the present disclosure.

FIG. 1F is a side view of the clot retrieval device of FIGS. 1A and 1B upon transitioning to a clot pinching configuration, in accordance with the present disclosure.

FIG. 1G is an additional side view of the clot retrieval device in the clot pinching configuration illustrated in FIG. 1F, in accordance with the present disclosure.

FIG. 2A is a side view of an additional example clot retrieval device in a clot engaging configuration, in accordance with the present disclosure.

FIG. 2B is a cross-sectional view of a proximal portion of the clot retrieval device of FIG. 2A, in accordance with the present disclosure.

FIGS. 2C-2E illustrate examples of a third body of the clot removal device of FIG. 2A, in accordance with the present disclosure.

FIG. 2F is an additional side view of the clot retrieval device of FIG. 2A, in accordance with the present disclosure.

FIG. 2G illustrates an example third collar of the clot removal device of FIG. 2A, in accordance with the present disclosure.

FIG. 3A illustrates an additional example clot retrieval device having a spring in a compressed configuration, in accordance with the present disclosure.

FIG. 3B illustrates the clot retrieval device of FIG. 3A where the spring is in an elongated configuration, in accordance with the present disclosure.

FIG. 4 is a flow diagram outlining a method of capturing a clot using the clot retrieval device of FIGS. 1A through 1G.

FIG. 5 is a flow diagram outlining a method of capturing a clot using the clot retrieval device of FIGS. 2A through 2G.

### DETAILED DESCRIPTION

The design and functionality described in this application is intended to be exemplary in nature and is not intended to limit the instant disclosure in any way. Those having skill in the pertinent art will appreciate that the teachings of the disclosure may be implemented in a variety of suitable forms, including those forms disclosed herein and additional forms known to those having skill in the art pertinent.

It will be apparent from the foregoing description that, while particular embodiments of the present disclosure have been illustrated and described, various modifications can be made without departing from the spirit and scope of the disclosure. For example, while the embodiments described herein refer to particular features, the disclosure includes embodiments having different combinations of features. The disclosure also includes embodiments that do not include all of the specific features described. Specific embodiments of the present disclosure are now described in detail with reference to the figures, wherein identical reference numbers indicate identical or functionality similar elements.

The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

In the following description, numerous specific details are set forth. But it is to be understood that examples of the disclosed technology can be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” “one example,” “an example,” “some examples,” “certain examples,” “various examples,” etc., indicate that the embodiment(s) and/or example(s) of the disclosed technology so described may include a particular feature, structure, or characteristic, but not every embodiment necessarily includes the particular feature, structure, or characteristic. Further, repeated use of the phrase “in one embodiment” or the like does not necessarily refer to the same embodiment, example, or implementation, although it may.

By “comprising” or “containing” or “including” or “having” is meant that at least the named compound, element, particle, configuration, or method step is present in the composition or device or method, but does not exclude the presence of other compounds, materials, particles, method steps, or configurations even if the other such compounds, material, particles, method steps, or configurations have the same function as what is named.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

Unless otherwise specified, the use of the ordinal adjectives “first,” “second,” “third,” etc., to describe a common object, merely indicate that different instances of like objects are being referred to, and are not intended to imply that the objects so described should be in a given sequence, either temporally, spatially, in ranking, or in any other manner.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human (e.g., rat, dog, pig, monkey, or the like).

Accessing the various vessels within the vasculature, whether they are coronary, pulmonary, or cerebral, involves well-known procedural steps and the use of a number of conventional, commercially-available accessory products. These products, such as angiographic materials and guidewires are widely used in laboratory and medical procedures. When these products are employed in conjunction with the system and methods of this disclosure in the description below, their function and exact constitution are not described in detail.

The disclosed technology can generally include a clot removal device having a sliding cage (e.g., a first body) and an outer cage (e.g., a second body) radially surrounding the sliding cage. A pull wire can be affixed to the sliding cage such that upon tension being applied to the pull wire, the sliding cage can displace proximally and independently of the outer cage. Upon the sliding cage displacing proximally, the sliding cage and the outer cage can pinch the clot. In some instances, the clot removal device can further include an inner channel (e.g., third body). The outer cage and the sliding cage can radially surround the inner channel. Upon tension being applied, the inner channel can move proximally and independently of the outer cage and sliding cage. Subsequently, the sliding cage can move proximally in relation to the outer cage and independently of the outer cage. In such configuration the sliding cage and the outer cage can further pinch the clot and the clot can thereby become further integrated within the clot removal device. Accordingly, the efficient and effective removal of a clot from a blood vessel can be performed.

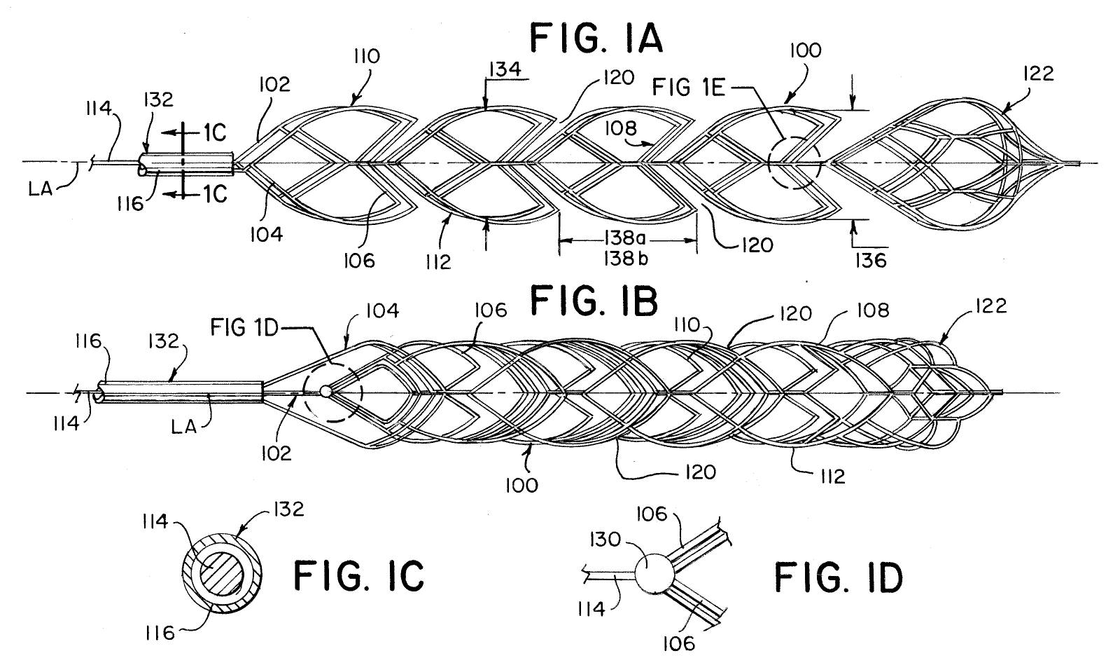

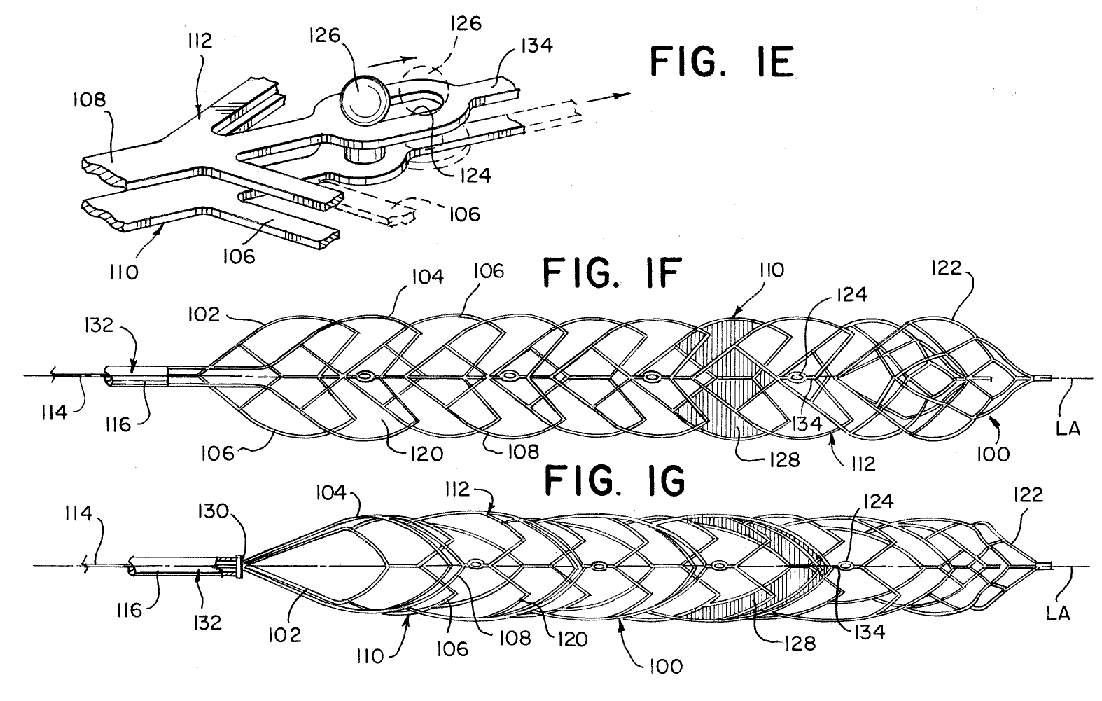

Referring now to the Figures, FIGS. 1A and 1B illustrate side views of an example clot retrieval device 100 in a clot engaging configuration. The clot retrieval device 100 can include a first expandable framework 102 and a second expandable framework 104. The first expandable framework 102 can include a first plurality of struts 106 and the second expandable framework 104 can include a second plurality of struts 108. The clot retrieval device 100 can have a longitudinal axis LA.

The first expandable framework 102 and the second expandable framework 104 can be collapsible into a restraining sheath (e.g., a microcatheter) sized to traverse a clot or other obstruction. The clot retrieval device 100 can be positioned proximate the clot in a blood vessel. Optionally, the clot retrieval device 100 can traverse the clot such that a portion of the clot remove device 100 is forward in relation to the clot. The first expandable framework 102 and the second expandable framework 104 can each be configured to self-expand upon release from the restraining sheath. Upon release, the clot retrieval device 100 can transition from a constrained delivery configuration to the clot engaging configuration such that the clot retrieval device 100 can be subsequently used to facilitate clot removal, flow restoration, and or fragmentation protection.

Upon transitioning to the clot engaging configuration, the first plurality of struts 106 of the first expandable framework 102 can expand to form a first body 110. Similarly, the second plurality of struts 108 of the second expandable framework 104 can expand to form a second body 112. The second body 112 can at least partially radially surround the first body 110. Optionally, the second body 112 can entirely radially surround the first body 110.

Both the first expandable framework 102, including the first plurality of struts 106, and the second expandable framework 104, including the second plurality of struts 108, can preferably be made from a material capable of recovering its shape automatically once released from the constrained delivery configuration. A super-elastic or pseudo-elastic material such as Nitinol or an alloy of similar properties is particularly suitable. The material can have a high recoverable strain sufficient to resiliently collapse and expand as described herein. The material could be in many forms such as wire or strip or sheet or tube. A particularly suitable manufacturing process is to laser cut a Nitinol tube and then heat set and electropolish the resultant structure to create a framework of struts and connecting elements. For example, the first expandable framework 102 and the second expandable framework 104 can each be laser cut from a Nitinol tube having an outer diameter of approximately 0.40 millimeters. Each of the cells in the first and second expandable frameworks 102, 104 can be any of a range of shapes as understood by a person skilled in the pertinent art according to the teachings disclosed herein. The first and second expandable frameworks 102, 104 can be rendered visible under fluoroscopy through the addition of alloying elements or through a variety of other coatings or marker bands. For instance, the first and second expandable frameworks 102, 104 can include material and/or markers with radiopaque material including, but not limited to Barium Sulphate, Bismuth SubCarbonate, Barium OxyChloride, Gold, Tungsten, Platinum, Iridium, Tantalum, and alloys thereof. Specifically, in some examples, the first and second expandable frameworks 102, 104 can include radiopaque markers having an Iridium alloy, and more specifically a Platinum-Iridium alloy.

As illustrated in FIGS. 1A and 1B, in the clot engaging configuration, the first body 110 and the second body 112 can each have a substantially cylindrical shape. Further, the first body 110 can have a first inner diameter 134 and the second body 112 can have a second inner diameter 136. The first inner diameter 134 can be approximately the same as the second inner diameter 136 such that the first body 110 and the second body 112 can substantially align with each other. By way of example, the first inner diameter 134 of the first body can be approximately 4.75 millimeters and the second inner diameter 136 can be approximately 5 millimeters. Because the first body 110 and the second body 112 have substantially equal inner diameters 132, 134, the first body 110 can exert an outward force onto the second body 112 in the clot engaging configuration.

FIGS. 1A and 1B illustrate the first body 110 and the second body 112 in a first position. The first plurality of struts 106 can form a first plurality of scaffolding segments 138a having closed cells and the second plurality of struts 108 of the second body 112 can form a second plurality of scaffolding segments 138b also having closed cells. The first plurality of scaffolding segments 138a and the second plurality of scaffolding segments 138b can be substantially aligned with each other. A gap can be formed between each scaffolding segment of the first and second plurality of scaffolding segments 138a, 138b. Such gap can be a clot reception space 120 configured to receive a clot. Portions of the clot can enter such clot reception spaces 120, thereby being captured by the clot retrieval device 100, upon the first body 110 moving from the first position to a second position in relation to the second body 112 as further discussed herein.

A distal end of the first body 110 and the second body 112 can form a distal basket 122. The distal basket 122 can have a substantially conical shape and can mitigate and/or prevent captured fragments of a clot from migrating out of the clot retrieval device 100.

As further illustrated in FIGS. 1C and 1D, a proximal end of the clot retrieval device 100 can include a shaft 132 including a pull wire 114 surrounded by an outer sheath 116. Proximal struts of the first plurality of struts 106 can be affixed to the pull wire 114, as illustrated in FIG. 1D. The pull wire 114 can be made of stainless steel, MP35N, Nitinol, or other material of suitably high modulus and tensile strength. The pull wire 114 can preferably have a solid core but can also have a hollow core. The first plurality of struts 106 of the first body 110 can be affixed to the pull wire 114 at a joint 130 via welding, bonding, by virtue of being cut from a contiguous tube, or other means of attachment. The joint 130 can be created at the approximate location of attachment of the proximal struts of the first plurality of struts 106 and the pull wire 114. Such joint 130 can inhibit unintended movement beyond a desired position when the first body 110 moves in relation to the second body 112 as further discussed herein. The second plurality of struts 108 of the second body 112 can be joined to the outer sheath 116, via welding, bonding, or the second body 112 can be formed using the same Nitinol or other material tubing of the outer sheath 116. Because the first body 110 and the second body 112 are affixed to independent portions of the shaft 132 (e.g., the pull wire 114 and the outer sheath 116, respectively), the first body 110 and the second body 112 can move independently of each other upon tension being applied to the pull wire 114 as further discussed herein.

FIG. 1E illustrates an expanded view of the substantially aligned first body 110 and the second body 112 and including an optional radially extending strut 126 and eyelet 124 to inhibit range of sliding movement between the first body 110 and the second body 112. As illustrated, the second body 112 can radially surround the first body 110 such that the second plurality of struts 108 are exterior to the first plurality of struts 106. The second plurality of struts 108 can further include one or more eyelets 124, and the first plurality of struts 106 can include one or more radially extending “connector” struts each extending through a respective eyelet 124. Each eyelet 124 can have an elongated or alternatively shaped opening. By way of example, the eyelet 124 can be substantially ovular, circular, rectangular, or the like. In some example, the eyelet 124 can be substantially tapered.

FIGS. 1F and 1G illustrate side views of the clot retrieval device 100 in a clot pinching configuration in which the first body 110 moves (e.g., slides) from the first position as illustrated in FIGS. 1A and 1B to a second position in relation to the second body 112. The pull wire 114 can be pulled in the proximal direction to apply tension. The tension can cause the first body 110 to move from the first position (FIGS. 1A and 1B) to the second position (FIGS. 1F and 1G) in relation to the second body 112. Accordingly, the first body 110 can slide from the first position to the second position in relation to the second body 112 such that the first body 110 and the second body 112 become engaged with one another. For example, the first body 110 can slide less than approximately 5 millimeters in relation to the second body 112. Optionally, the first body 110 can slide less than approximately 4 millimeters in relation to the second body 112. Optionally, the first body 110 can slide less than approximately 2 millimeters in relation to the second body 112. Optionally, the first body 110 can slide less than approximately 0.5 millimeters in relation to the second body 112. When the first body 110 moves from the first position to the second position and the device 100 includes one or more radially extending struts 126 through respective eyelets 124 as illustrated in FIG. 1E, the radially extending struts 126 can engage with the eyelets 124, thereby allowing the first body 110 and the second body 112 to become engaged with one another. Further, the eyelet 124 can inhibit the first body 110 from moving, in relation to the second body 112, beyond the second position, as the eyelet 124 can restrict the radially extending strut 126 from moving too far in the proximal direction.

Additionally, or alternatively, the first expandable framework 102 and the second expandable framework 104 can be coated with a polymer coating (e.g., parylene) to temporarily hold the first body 110 in the first position in relation to the second body 112. Upon the pull wire 114 being pulled in the proximal direction, the polymer coating can fail such that the first body 110 can move from the first position to the second position in relation to the second body 112. Aspiration can be applied in order to remove particulate from the failed polymer coating.

Additionally, or alternatively, a shape memory effect of the first body 110 and the second body 112 can be used to cause automatic displacement of the first body 110 after a predetermined time period has lapsed. For example, the second plurality of struts 108 of the second expandable framework 104 can be heat treated locally to increase the austenite finish temperature to a range greater than a typical body temperature during a stroke or other critical body occurrence. The second plurality of struts 108 can expand upon being re-sheathed then be heated to the austenite finish temperature by electrical current. Upon the austenite finish temperature being reached, the first body 110 can automatic move (e.g., slide) in relation to the second body 112 from the first position to the second position, such that the clot can be pinched between the first body 110 and the second body 112.

The pull wire 114 can be pulled in the proximal direction such that the first body 110 moves (e.g., slides) in the proximal direction until the proximal struts of the first plurality of struts 106 encounter the joint 130 positioned proximate the shaft 132. As such, the joint 130 can act as a mechanism to prevent undesired movement beyond the second position. As the first body 110 moves from the first position to the second position the average cross-sectional area of the plurality of clot reception spaces 120 can decrease (e.g., at least partially close). For example, the plurality of clot reception spaces 120 can at least partially close when the first plurality of scaffolding segments 138a slide in relation to the second plurality of scaffolding segments 138b such that the first plurality of scaffolding segment 138a become disposed across the clot reception spaces 120. Thereby, the clot can be pinched between the first body 110 and the second body 112. Pinching of the clot can prevent the clot from migrating out of the clot retrieval device 100, particularly upon retraction of the clot retrieval device 100, as the pinch can increase the grip of the clot retrieval device 100 as compared to other clot retrieval devices, particularly fibrin rich clots. Accordingly, the clot retrieval device 100 can ensure effective and efficient removal of the clot from the patient.

As illustrated in FIGS. 1F and 1G, the clot retrieval device 100 can include a polymer membrane 128 disposed between the first body 110 and the second body 112. The polymer membrane 128 (e.g., elastic membrane) can be configured to transition from a folded configuration when the first body 110 is in the first position such that the first body 110 and the second body 112 are disengaged to a stretched configuration upon the first body 110 moving from the first position to the second position. The polymer membrane 128 can thereby function to limit lateral movement of the first body 110 in relation to the second body 112 in addition to, or as an alternative to the radially extending strut 126 and eyelet 124 illustrated in FIG. 1E. The polymer membrane 128 can be formed by threading microfibers through the eyelets 124 of the second body 112, and/or the polymer membrane 128 can be formed by hooking the polymer membrane 128 into the eyelets 124. The polymer membrane 128 can prevent the clot from migrating out of the clot retrieval device 100 once the clot has been pinched between the first body 110 and the second body 112. Although FIGS. 1F and 1G illustrate the polymer membrane 128 in one location, it is contemplated that the clot retrieval device 100 can include additional polymer membranes 128 over multiple locations. For example, the clot retrieval device 100 can include a first polymer membrane proximate the distal basket of the 122 and a second polymer membrane proximate the shaft 132.

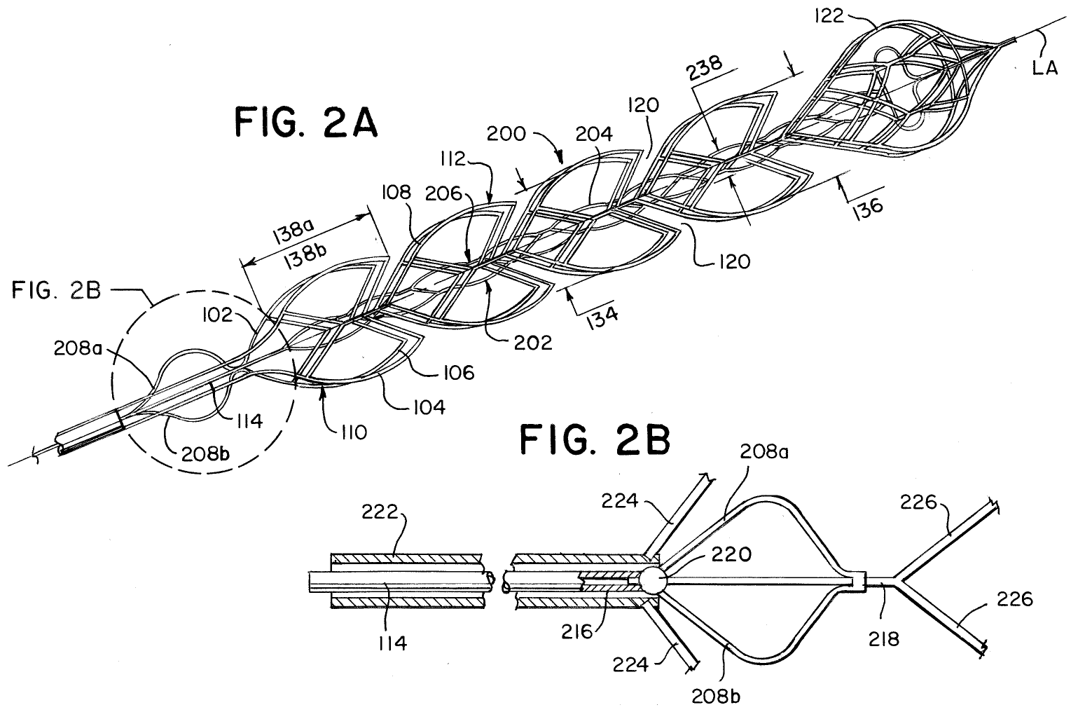

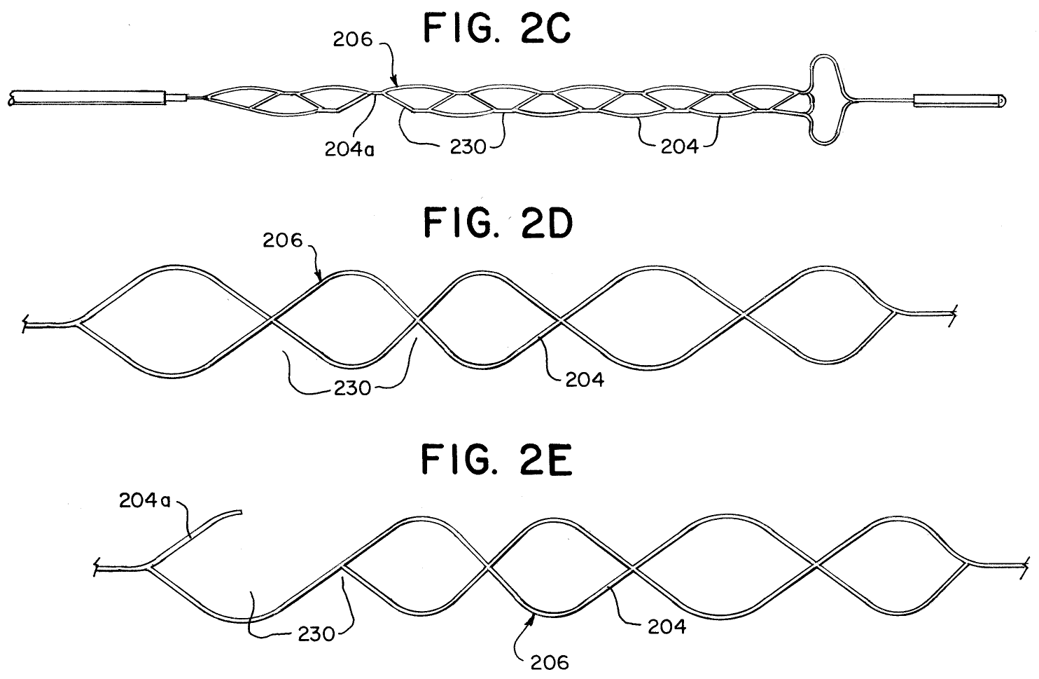

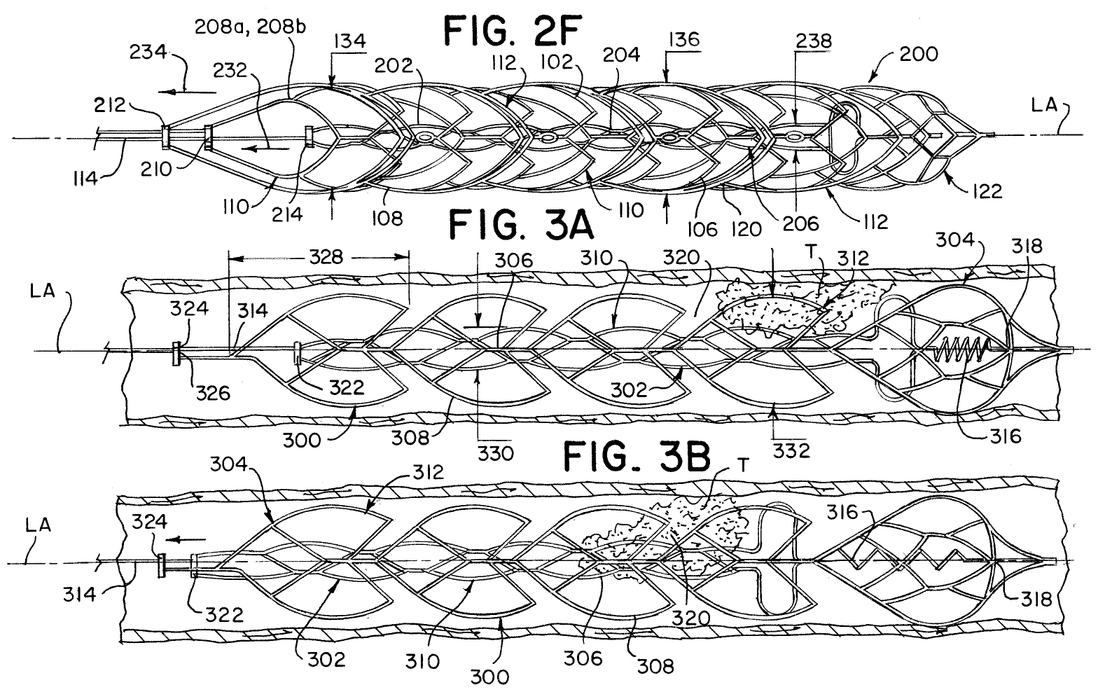

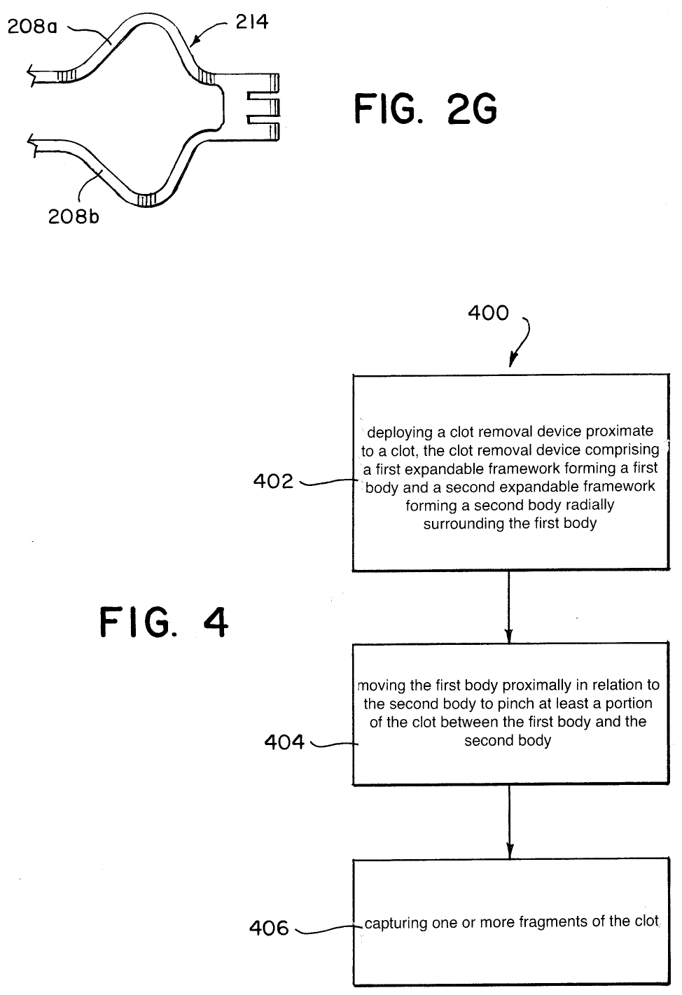

FIG. 2A illustrates an additional example clot retrieval device 200. As discussed above with reference to the clot retrieval device 100 illustrated in FIGS. 1A through 1G, the clot retrieval device 200 can similarly include the first expandable framework 102 including a first plurality of struts 106 and a second expandable framework 104 including a second plurality of struts 108. The clot retrieval device 200 can have a longitudinal axis LA. Upon the clot retrieval device 200 being deployed from a restraining sheath (e.g., microcatheter) and transitioning from a constrained delivery configuration to a clot engaging configuration, the first plurality of struts 106 of the first expandable framework 102 can self-expand to form the first body 110 and the second plurality of struts 108 of the second expandable framework 104 can self-expand to form the second body 112. The first body 110 and the second body 112 can be substantially cylindrical. Additionally, the first body 110 and the second body 112 can have substantially equal inner diameters 134, 136. As such, and as discussed above, the plurality of scaffolding sections 138a, 138b of the first body 110 and the second body 112 can be substantially aligned with one another.

In contrast to the clot retrieval device 100 illustrated in FIGS. 1A through 1G, the clot retrieval device 200 can further include a third expandable framework 202 having a third plurality of struts 204. Upon the clot retrieval device 200 being deployed from the restraining sheath, the third plurality of struts 204 of the third expandable framework 202 can self-expand to form a third body 206. The third body 206 can be substantially porous. Further, the third body 206 can similarly be substantially cylindrical and can have an inner diameter 238 that is less than the inner diameter 134 of the first body 110 and the inner diameter 136 second body 112. Thereby, the first body 110 and the second body 112 can radially surround the third body 206. Optionally, the third body 206 can have an inner diameter 238 that is approximately half (½) the size of the inner diameter 134 of the first body 110 and inner diameter 136 of the second body 112. Optionally, the third body 206 can have an inner diameter 238 that is approximately three quarters (M) the size of the inner diameter 134 of the first body 110 and the inner diameter 136 of the second body 112. The third expandable framework 202 can be preferably made from a material capable of recovering its shape automatically once released from a constricted delivery configuration. A super-elastic or pseudo-elastic material such as Nitinol or an alloy of similar properties is particularly suitable. The material can have a high recoverable strain sufficient to resiliently collapse and expand as described herein. The material could be in many forms such as wire or strip or sheet or tube. A particularly suitable manufacturing process is to laser cut a Nitinol tube and then heat set and electropolish the resultant structure to create a framework of struts and connecting elements. Optionally, the third expandable framework 202 can be laser cut from a Nitinol tube.

As discussed above with reference to the clot retrieval device 100, the first body 110 can include a first plurality of scaffolding segments 138a and the second body 112 can include a second plurality of scaffolding segments 138b. The first and second plurality of scaffolding segments 138a, 138b can substantially align with one another. A gap can be formed between each scaffolding segment of the first plurality and second plurality of scaffolding segments 138a, 138b. Such gap can be a clot reception space 120 configured to receive at least a portion of a clot upon the clot retrieval device 200 transitioning to a clot pinching configuration as further described herein. The configuration of the third body 206 can similarly create additional clot reception spaces 230 as further discussed herein.

The distal end of the clot retrieval device 200 can include the distal basket 122. The distal basket 122 can have a substantially conical shape and can mitigate captured fragments of a clot from migrating out of the clot retrieval device 200.

As further illustrated in FIG. 2B, a plurality of expanded struts 208a, 208b can be formed from the shaft 216 of the first body 110. For example, the shaft 216 of the first body 110 can be a tube (e.g., Nitinol tube) and the two expanded struts 208a, 208b can be formed (e.g., laser cut) from such tube. The shaft 216 of the first body 110 can be a hollow tube sized to receive the pull wire 114. As such, the pull wire 114 can extend through the shaft 216 of the first body 110. The plurality of expanded struts 208a, 208b can facilitate improved pinching when the clot retrieval device 200 transitions to the clot pinching configuration. Optionally, the plurality of expanded struts 208a, 208b can be used to allow re-expansion of the third body 206 to stabilize the clot if an effective pinch is not formed upon the clot retrieval device 200 transitioning to the clot pinching configuration. A shaft 222 of the second body 112 can surround the shaft 216 of the first body 110 and the pull wire 114. The shaft 222 of the second body 112 can be a tube (e.g., a Nitinol tube). Proximal struts 224 of the second body 112 can extend from a distal end of the shaft 222 of the second body 112. The proximal struts 224 can be the most proximal struts of the second expandable framework 104. A proximal end of a shaft 218 of the third body 206 can be affixed to a distal end of the pull wire 114. The shaft 218 of the third body 206 can be a solid core tube. A joint 220 (e.g. weld joint) can be formed where the shaft 218 of the third body 206 is affixed to the pull wire 114 and where the shaft 216 of the first body 110 forms the expanded struts 208a, 208b. Proximal struts 226 of the third body 206 can extend from a distal end of the shaft 218 of the third body 206. The proximal struts 226 can be the most proximal struts of the third expandable framework 202.

FIG. 2C through 2E illustrate various configurations of the third body 206. In FIGS. 2C through 2E, the third plurality of struts 204 can be configured to form a substantially cylindrical body of interconnected struts. The third plurality of struts 204 can be interconnected with one another such that the third plurality of struts form a majority of closed cells. The third plurality of struts 204 can be configured to form closed cells of differing sizes and shapes. Optionally, the closed cells can be sized according to the content of the clot to be captured (e.g., based on how soft and/or fibrin rich the clot to be captured is). As illustrated in FIGS. 2C through 2E, the third body 206 can further include a plurality of clot reception spaces 230. The size of each clot reception space 230 can be based on the configuration of the third plurality of struts 204. Optionally, as illustrated in FIGS. 2C and 2E, the third plurality of struts 204 can include at least one disconnected strut 204a. The disconnected strut can form an open cell. Such open cell can form a larger clot reception space 230, and thereby facilitate migration of the clot into the third body 206. Optionally, the third plurality of struts 204 can include a plurality of disconnected struts 204a such that a majority of the cells are open cells. The third body 206 can serve two primary functions. For example, the third body 206 can facilitate allowing blood to pass through so that there is at least partial blood flow through the blood vessel as a clot is being captured and withdrawn by the clot retrieval device 200. Additionally, upon the clot retrieval device 200 being deployed, a clot can become partially integrated into the clot reception spaces 120 of the first body 110 and the second body 112. Upon the third body 206 moving (e.g., sliding) in relation to the first body 110 and the second body 112, the third body 206 can interact with the clot, thereby promoting further integration of the clot between the first body 110 and the second body 112 prior to forming a pinch.

FIG. 2F illustrates an additional side view of the clot retrieval device 200. As illustrated in FIG. 2F, the first body 110 can include a first collar 210, the second body 112 can include a second collar 212, and the third body 206 can include a third collar 214. Each collar 210, 212, 214 can facilitate moving (e.g., sliding) the third body 206 in relation to the first body 110 and the second body 112 and subsequently moving (e.g., sliding) the first body 110 in relation to the second body 112 to pinch at least a portion of a clot. The pull wire 114 can be affixed to the third body 206 and can be threaded through each collar 210, 212, 214 such that upon the pull wire 114 being pulled in the proximal direction, the clot retrieval device 200 can move (e.g., slide) from a first position to a second position and subsequently from a second position to a third position.

FIG. 2G illustrates an example third collar 214. The third collar 214 can be disposed where the expanded struts 208a, 208b conjoin. The third collar 214 can be laser cut in a particular design to facilitate pinching the clot upon the clot retrieval device 200 moving from the first position to the second position and subsequently from the second position to the third position.

In order to pinch the clot using the clot retrieval device 200, the pull wire 114 can be pulled in a proximal direction. Such tension can cause the third body 206 to move (e.g., slide) in relation to the first body 110 and the second body 112 causing the clot retrieval device 200 to transition from the first position to the second position, as indicated by a first arrow 232. For example, the third body 206 can slide less than approximately 5 millimeters in relation to the first body 110 and the second body 112. Optionally, the third body 206 can slide less than approximately 4 millimeters in relation to the first body 110 and the second body 112. Optionally, the third body 206 can slide less than approximately 2 millimeters in relation to the first body 110 and the second body 112. Optionally, the third body 206 can slide less than approximately 0.5 millimeters in relation to the first body and the second body 112.

Upon the third body 206 moving in relation to the first body 110 and the second body 112, at least a portion of the clot can be pinched between the third body 206 and the first body 110 and the second body 112 to cause the portions of the clot to migrate inside the first body 110 and the second body 112. Additionally, upon the third body 206 moving from the first position to the second position, the third body 206 can become engaged with the first body 110 as the third collar 214 becomes engaged with the first collar 210. When the third collar 214 and the first collar 210 become engaged, the pull force can be transferred to the first body 110, thereby allowing for further displacement.

Subsequently, the first body 110 can move (e.g., slide) in relation to the second body 112 such that the clot retrieval device 200 transitions from the second position to the third position, as indicated by a second arrow 234. For example, the first body 110 can slide less than approximately 5 millimeters in relation to the second body 112. Optionally, the first body 110 can slide less than approximately 4 millimeters in relation to the second body 112. Optionally, the first body 110 can slide less than approximately 2 millimeters in relation to the second body 112. Optionally, the first body 110 can slide less than approximately 0.5 millimeters in relation to the second body 112.

Upon the first body 110 moving from the second position to the third position, the first body 110 engaged with the third body 206 can become engaged with the second body 112 causing an average cross-sectional area of the plurality of clot reception spaces 120 to decrease (e.g., at least partially close). Thereby, at least a portion of the clot can be further pinched. The portions of the clot can be pinched between the first body 110 and the second body 112 and, additionally, the third body 206. Accordingly, the portions of the clot can further migrate inside third body 206. As such, the portions of the clot can further migrate into the clot reception spaces 230 of the third body 206. Upon transitioning to the third position, the expanded struts 208a, 208b can become resheathed as the first collar 210 becomes engaged with the second collar 212. When the first collar 210 becomes engaged with the second collar 212, the pull force can be transferred to the second body 112. Accordingly, the first body 110, the second body 112, and the third body 206, including the captured clot or portions thereof, can be removed simultaneously from the patient's vasculature.

Optionally, the clot retrieval device 200 can include a seal that allows the clot retrieval device 200 to maintain a pinch even if the physician stops applying tension. For example, the clot retrieval device 200 can include a seal disposed proximate the weld joint 220. The seal can maintain the clot retrieval device 200 in place using friction. As such, the seal can create enough static friction such that the clot removal device 200 does not displace when a physician stops applying tension while also ensuring the static friction is not too high that a user cannot overcome such static friction when manipulating the clot retrieval device 200 during retrieval of the clot and removal of the clot retrieval device 200 from a patient.

FIGS. 3A and 3B illustrate an additional example clot retrieval device 300. The clot retrieval device 300 can have a constrained delivery configuration and a clot engaging configuration and can be configured to remove a clot (e.g., thrombus) T from a blood vessel. The clot removal device 300 can have a longitudinal axis LA. The clot removal device 300 can be in the constrained delivery configuration when the clot removal device 300 is positioned within a restraining sheath (e.g., microcatheter). Upon the restraining sheath being retracted, the clot removal device 300 can transition to the clot engaging configuration. The clot retrieval device 300 can include an inner expandable framework 302 and an outer expandable framework 304. The inner expandable framework 302 can include an inner plurality of struts 306 that self-expand to form an inner body 310 upon the clot retrieval device 300 transitioning from the constrained delivery configuration to the clot engaging configuration. Similarly, the outer expandable framework 304 can include an outer plurality of struts 308 that self-expand form an outer body 312 upon the clot retrieval device 300 transitioning from the constrained delivery configuration to the clot engaging configuration. The inner expandable framework 302 and the outer expandable framework 304 can be preferably made from a material capable of recovering its shape automatically once released from a constricted delivery configuration and further include the additional characteristics as described above with reference to the first expandable framework 102 and the second expandable framework 104.

The inner body 310 and the outer body 312 can have different inner diameters 330, 332 and/or configurations. For example, the inner body 310 can have a smaller inner diameter 330 than the inner diameter 332 of the outer body 312, as such the outer body 312 can radially surround the inner body 310. Optionally, the inner diameter 330 of the inner body 310 can be approximately half of the size of the inner diameter 332 of the outer body 312. Optionally, the inner diameter 330 of the inner body 310 can be approximately ¾ of the size of the inner diameter 332 of the outer body 312. As illustrated in FIG. 3A, the differing diameters 330, 332 and shape configurations of the inner body 310 and the outer body 312 can form a plurality of clot reception spaces 320 configured to engage with a clot T. For example, the outer body 312 can include a plurality of scaffolding sections. A clot reception space 320 can be formed between each scaffolding section 338 of the plurality of scaffolding sections. Additionally, the inner body 310 can have a substantially “S” wave shape. Such “S” wave shape can facilitate pinching and capturing the clot upon the inner body 310 moving in relation to the outer body 312, and upon capturing the clot, preventing the captured clot from migrating out of the clot retrieval device 300.

The inner body 310 and the outer body 312 can each be affixed to a pull wire 314. The pull wire 314 can include a first stopper 322 and a second stopper 324 at a proximal end 326 of the pull wire. The first stopper 322 can be disposed distally in relation to the second stopper 324.

A spring 316 can be affixed to a distal end 318 of the pull wire 314. The spring 316 can be configured to transition from a compressed configuration and an elongated configuration upon actuation. The spring 316 can be configured to maintain a central position of the inner body 310 within the outer body 312. In the previously illustrated clot retrieval devices 100, 200 because the first body 110 has an approximately equal inner diameter 134 to the second body 112, the first body 110 resides centrally within the second body 112 because of outward force from the first body 110 onto the second body 112. When the inner body 310 has a substantially smaller inner diameter 330 than the outer body 312 as illustrated in FIGS. 3A and 3B, a distal portion of the inner body 310 can radially deflect with respect to the outer body 312 but for the spring 316 which inhibits the distal portion from deflecting. The spring 316 can further function to hold the device 300 in a first position when the pull wire 314 is not under tension.

As illustrated in FIG. 3A, the clot retrieval device 300 can be positioned proximate to the clot T. At least a portion of the clot retrieval device 300 can traverse the clot T such that a distal end of the clot retrieval device 300 can be forward in relation to the clot T. The pull wire 314 can be pulled in a proximal direction causing the inner body 310 to move (e.g., slide) from the first position to the second position in relation to the outer body 312.

FIG. 3B illustrates the clot retrieval device 300 transitioning to a clot pinching configuration upon the inner body 310 moving from the first position to the second position. The pull wire 314 can be pulled in the proximal direction until the first stopper 322 is proximate and/or engages (e.g., touches or becomes within a predetermined distance of the second stopper 324) the second stopper 324, as such the second stopper 324 can serve as an indicator of when to stop applying tension to the pull wire 314 and/or gradually weaken the amount of tension being applied to the pull wire 314. When the pull wire 314 is pulled in the proximal direction, the spring 316 can be actuated, causing the spring 316 to transition from the compressed configuration to the elongated configuration. When the spring 316 transitions to the elongated configuration, the inner body 310 can move from the first position to the second position. Such movement from the first position to the second position can cause the average cross-sectional area of the clot reception spaces 320 to decrease (e.g., at least partially close), thereby pinching the clot T between the inner body 310 and the outer body 312. Upon pinching the clot T between the inner body 310 and outer body 312, the inner body 310 and the outer body 312, including the clot T, can be retracted into the restraining sheath (e.g., microcatheter), and subsequently the clot retrieval device 300 can be removed from the patient.

FIG. 4 illustrates a flow diagram outlining a method 400 of capturing a clot using the clot retrieval device 100 illustrated in FIGS. 1A through 1G. The method 400 can include deploying 402 the clot retrieval device 100 proximate to the clot. As discussed herein, the clot retrieval device 100 can include a first expandable framework 102 and a second expandable framework 104. Upon deploying the clot retrieval device 100, the clot retrieval device 100 can transition from a constrained delivery configuration to a clot engaging configuration and the first expandable framework 102 can expand to form a first body 110 while the second expandable framework 104 can expand to form a second body 112 that radially surrounds the first body 110. The first body 110 and the second body 112 can have substantially the same inner diameter 134. 136, such that the first body 110 and the second body 112 substantially radially align with one another.

The method 400 can further include moving 404 (e.g., sliding) the first body 110 proximally in relation to the second body 112 to pinch at least a portion of the clot between the first body 110 and the second body 112 such that the clot removal device 100 transitions to a clot pinching configuration. By way of example, tension can be applied to the pull wire 114 until the first body 110 encounters the joint 130. As the pull wire 114 is pulled in the proximal direction, the first body 110 can move in the proximal direction in relation to the second body 112, thereby pinching at least a portion of the clot between the first body 110 and the second body 112.

The method 400 can further include capturing 406 one or more fragments of the clot.

Additionally, the method 400 can include retracting the first body 110 and the second body 112 simultaneously, as the first body 110 and the second body 112 become engaged upon the first body 110 moving in relation to the second body 112. Similarly, upon the first body 110 and the second body 112 being retracted into a restraining sheath, the restraining sheath can be removed from a patient's vasculature.

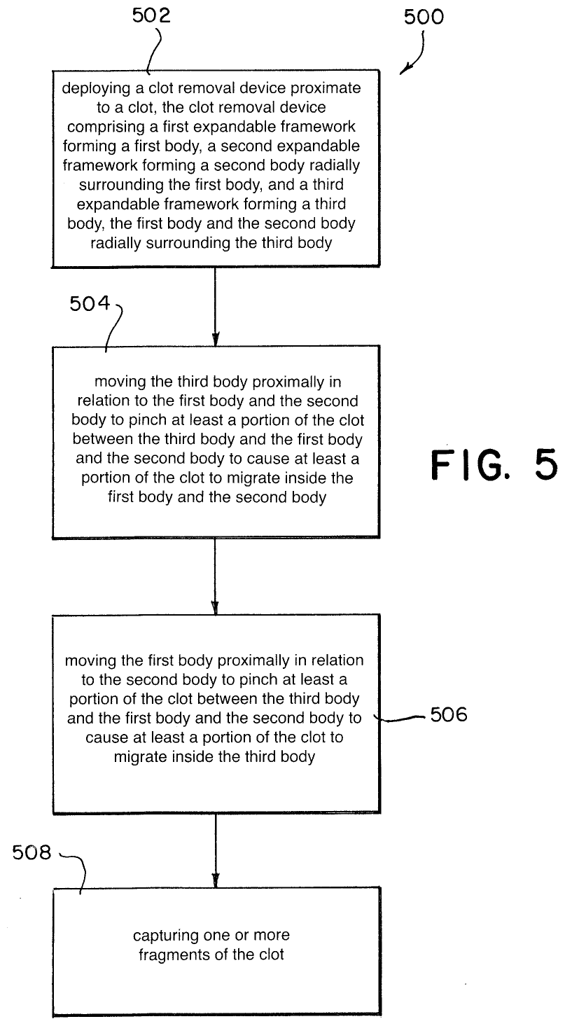

FIG. 5 illustrates a flow diagram outlining a method 500 of capturing a clot using the clot retrieval device 200 illustrated in FIGS. 2A through 2F. The method 500 can include deploying 502 the clot retrieval device 200 proximate to the clot. As discussed herein, the clot retrieval device 100 can include a first expandable framework 102, a second expandable framework 104, and a third expandable framework 202. Upon deploying the clot retrieval device 200, the clot retrieval device 200 can transition from a constrained delivery configuration to a clot engaging configuration and the first expandable framework 102 can expand to form a first body 110 while the second expandable framework 104 can expand to form a second body 112 that radially surrounds the first body 110. The first body 110 and the second body 112 can have substantially the same inner diameter 134. 136, such that the first body 110 and the second body 112 can substantially align with each other. Similarly, the third expandable framework 202 can expand to form the third body 206. The first body 110 and the second body 112 can radially surround the third body 206, as the third body 206 can have a smaller inner diameter 238 as compared to the first body 110 and the second body 112.

The method 500 can further include moving 504 (e.g., sliding) the third body 206 in relation to the first body 110 and the second body 112 to pinch at least a portion of the clot between the third body 206 and the first body 110 and the second body 112 such that the clot removal device 200 transitions to a clot pinching configuration. As such, at least a portion of the clot can migrate into the first body 110 and the second body 112. Upon moving the third body 206 in relation to the first body 110 and the second body 112, the third body 206 can become engaged with the first body 110.

The method 500 can further include moving 506 (e.g., sliding) the first body 110 proximally in relation to the second body 112 to further pinch at least a portion of the clot between the third body 206 and the first body 110 and the second body 112. As the first body 110 moves in relation to the second body 112, the average cross-sectional area of the clot reception spaces 120 can decrease, thereby pinching at least a portion of clot and preventing and/or mitigating the captured portions of the clot from migrating back out of the clot retrieval device 200. Upon pinching the clot, at least a portion of the clot can migrate inside the third body 206. When at least a portion of the clot is within third body 206, the potential for the portions of the captured clot to migrate back out of the clot retrieval device 200 can decrease.

The method 500 can further include capturing 508 one or more fragments of the clot.

Additionally, the method 500 can include simultaneously retracting the first body 110, the second body 112, and the third body 206. Upon being retracted into a delivery microcatheter, the delivery microcatheter can be removed from a patient's vasculature, and thus, the clot can be removed effectively and efficiently from the patient's vasculature.

Certain examples and implementations of the disclosed technology are described above with reference to block and flow diagrams according to examples of the disclosed technology. It will be understood that one or more blocks of the block diagrams and flow diagrams, and combinations of blocks in the block diagrams and flow diagrams, respectively. Likewise, some blocks of the block diagrams and flow diagrams do not necessarily need to be performed in the order presented, can be repeated, or do not necessarily need to be performed at all, according to some examples or implementations of the disclosed technology. It is also to be understood that the mention of one or more method steps does not preclude the presence of additional method steps or intervening method steps between those steps expressly identified. Additionally, method steps from one process flow diagram or block diagram can be combined with method steps from another process diagram or block diagram. These combinations and/or modifications are contemplated herein.

What is claimed is:

1. A clot retrieval device comprising a constrained delivery configuration and a clot engaging configuration and being configured to remove a clot from a blood vessel, the device comprising:
a first expandable framework comprising a closed distal end, a closed proximal end, and a first plurality of struts forming a substantially cylindrical first body comprising a plurality of cells having two or more different shapes;
a second expandable framework comprising a closed distal end, a closed proximal end, and a second plurality of struts forming a substantially cylindrical second body comprising a plurality of cells having two or more different shapes, a length of the second body at least partially surrounding and substantially aligned with a length of the first body; and
a third expandable framework comprising a third plurality of struts forming a third body, the first body and the second body at least partially surrounding the third body in the clot engaging configuration,
wherein in the clot engaging configuration, the first body is configured to move proximally from a first position to a second position in relation to the second body to compress at least a portion of the clot between the plurality of cells of the first body and the plurality of cells of the second body, and
wherein the third plurality of struts includes at least one disconnected strut.

2. The clot retrieval device of claim 1, wherein the first body has a first inner diameter and the second body has a second inner diameter, the first inner diameter and the second inner diameter being substantially equal.

3. The clot retrieval device of claim 1, wherein when the first body is in the first position, the first plurality of struts and the second plurality of struts are disengaged such that a plurality of clot reception spaces are formed.

4. The clot retrieval device of claim 3, wherein when the first body is in the second position, the first plurality of struts and the second plurality of struts are engaged such that an average cross-sectional area of the plurality of clot reception spaces decreases upon movement of the first body from the first position to the second position.

5. The clot retrieval device of claim 1, wherein the first plurality of struts comprises at least one radially extending strut and the second plurality of struts comprises at least one eyelet through which the at least one radially extending strut radially extends, the at least one eyelet and the at least one radially extending strut being configured such that when the first body moves from the first position to the second position, the at least one radially extending strut engages the at least one eyelet to inhibit the first plurality of struts from moving, in relation to the second plurality of struts, beyond the second position.

6. The clot retrieval device of claim 5, wherein each eyelet is tapered.

7. The clot retrieval device of claim 1, further comprising a polymer coating to engage the first plurality of struts and the second plurality of struts, the polymer coating configured to fail to allow the first body to move from the first position to the second position.

8. The clot retrieval device of claim 1, further comprising at least one polymer membrane, wherein a first polymer membrane is affixed to the first plurality of struts and the second plurality of struts such that the first polymer membrane is disposed between the first body and the second body.

9. The clot retrieval device of claim 8, wherein the first polymer membrane is in a folded configuration when the first body is in the first position and the first polymer membrane transitions to a stretched configuration when the first body moves proximally to the second position.

10. The clot retrieval device of claim 1, wherein the third body is configured to move in relation to the first body and the second body.

11. The clot retrieval device of claim 1, wherein a proximal end of the clot retrieval device includes a plurality of expanded struts that form a collar.

12. The clot retrieval device of claim 1, wherein the third body comprises a plurality of clot reception spaces, the plurality of clot reception spaces configured to engage the clot.

13. A method of capturing a clot, the method comprising:
deploying a clot retrieval device proximate to the clot, the clot retrieval device comprising:
a first expandable framework comprising a closed distal end, a closed proximal end, and a first plurality of struts forming a first substantially cylindrical body comprising a plurality of cells having two or more different shapes;
a second expandable framework comprising a closed distal end, a closed proximal end, and a second plurality of struts forming a second substantially cylindrical body comprising a plurality of cells having two or more different shapes, a length of the second body at least partially surrounding and substantially aligned with a length of the first body; and
a third expandable framework comprising a third plurality of struts forming a third body, the first body and the second body at least partially surrounding the third body in a clot engaging configuration, the third plurality of struts including at least one disconnected strut,
moving the first body proximally in the clot engaging configuration in relation to the second body to pinch at least a portion of the clot between the plurality of cells of the first body and the plurality of cells of the second body; and
capturing one or more fragments of the clot.

14. The method of claim 13, wherein moving the first body in relation to the second body to pinch at least a portion of the clot between the first body and the second body includes applying tension to a pull wire, the pull wire being in mechanical communication with the first body.

15. The method of claim 13, further comprising retracting the first body and the second body simultaneously.

16. The method of claim 13 further comprising:
retracting the third body in a proximal direction to engage the first body and the second body.
